(19) United States
(12) Patent Application Publication
(10) Pub. No.: US 2023/0059203 A1
(43) Pub. Date: Feb. 23, 2023

(54) RNA Detection

(71) Applicant: Akoya Biosciences, Inc., Menlo Park, CA (US)

(72) Inventors: Julia Kennedy-Darling, Redwood City, CA (US); Peter J. Miller, Cambridge, MA (US)

(21) Appl. No.: 17/787,075

(22) PCT Filed: Dec. 21, 2020
(86) PCT No.: PCT/US2020/066400

§ 371 Date: Jun. 17, 2022

Related U.S. Application Data
(60) Provisional application No. 62/950,928, filed on Dec. 19, 2019.

(51) Int. Cl. C12Q 1/682; C12Q 1/6841; C12Q 1/6876

(52) U.S. Cl. CPC C12Q 1/682; C12Q 1/6841; C12Q 1/6876; C12Q 2600/178

(57) ABSTRACT

Methods for RNA detection in biological samples include (a) contacting a biological sample with a first composition featuring multiple different types of unlabeled oligonucleotide probes that hybridize to RNA species in the sample; (b) contacting the biological sample with a hybridization agent featuring a chaotropic compound; (c) contacting the biological sample with a second composition that includes multiple different types of labeled oligonucleotide probes, where each of the different types of labeled oligonucleotide probes selectively hybridizes to one of the different types of unlabeled oligonucleotide probes; (d) obtaining at least one image of the biological sample with the multiple different types of labeled oligonucleotide probes bound to the sample; and (e) identifying spatial locations of the RNA species in the sample based on components of the at least one image that correspond to the different types of labeled oligonucleotide probes, where the biological sample is contacted with the second composition under isothermal conditions.

### CROSS-REFERENCE TO RELATED APPLICATIONS

This application claims priority to U.S. Provisional Application No. 62/950,928, the entire contents of which are incorporated herein by reference.

### TECHNICAL FIELD

This disclosure relates to the detection of RNA in biological samples, and compositions, methods, and systems for sample analysis.

### BACKGROUND

In situ detection of RNA and RNA-related species is critical for understanding native sample environments and measuring key biological indicators and parameters. Certain in-situ assays are limited to the detection of single RNA target species. Some assays involve co-detection of multiple RNA species by controlling the hybridization state and heating the sample to perform specific hybridization of RNA probes.

### SUMMARY

The present disclosure features compositions, methods, and systems for performing multiplexed RNA detection in a variety of different types of biological samples. In particular, some compositions are formulated to allow hybridization of dye-labeled olignonucleotide probes to oligonucleotide-labeled RNA species in a sample without changing the sample temperature. By eliminating sample heating steps, the analytical workflow is considerably simplified. Compositions that correspond to buffer solutions, and which contain one or more chaotropic components, can be used to control the hybridization and dehybridization states of oligonucleotide-labeled RNA species in the sample, thereby either causing hybridization of dye-labeled oligonucleotide probes to the sample for detection, or dehybridization of dye-labeled oligonucleotide probes from the sample to prepare for additional analytical cycles. In a single analytical cycle, a biological sample can be labeled with multiple different dye-labeled RNA probes, and RNA species corresponding to each of the different dye-labeled RNA probes can be quantified. The detection of RNA species can be repeated for multiple cycles with batches of different dye-labeled RNA probes, allowing a large number of RNA species in the sample to be detected.

The disclosure features methods that include: (a) contacting a biological sample with a first composition featuring multiple different types of unlabeled oligonucleotide probes that hybridize to RNA species in the sample; (b) contacting the biological sample with a hybridization agent featuring a chaotropic compound; (c) contacting the biological sample with a second composition featuring multiple different types of labeled oligonucleotide probes, where each of the different types of labeled oligonucleotide probes selectively hybridizes to one of the different types of unlabeled oligonucleotide probes; (d) obtaining at least one image of the biological sample with the multiple different types of labeled oligonucleotide probes bound to the sample; and (e) identifying spatial locations of the RNA species in the sample based on components of the at least one image that correspond to the different types of labeled oligonucleotide probes, where the biological sample is contacted with the second composition under isothermal conditions.

Embodiments of the methods can include any one or more of the following features.

The methods can include (f) removing the labeled oligonucleotide probes from the sample. The methods can include repeating steps (c)-(e) with a different set of labeled oligonucleotide probes to identify spatial locations of at least one additional RNA species in the sample. The methods can include repeating step (b) prior to repeating steps (c)-(e).

The methods can include, prior to repeating steps (c)-(e), contacting the biological sample with a third composition featuring multiple different types of unlabeled oligonucleotide probes that hybridize to RNA species in the sample, where the multiple different types of unlabeled oligonucleotide probes of the third composition are also present in the first composition.

The chaotropic compound can include dimethylsulfoxide and/or formamide. A weight percentage of the chaotropic compound in the hybridization agent can be between 5% and 20%.

The methods can include removing the labeled oligonucleotide probes from the sample by reductive cleavage and/or by enzymatic cleavage.

The methods can include removing the labeled oligonucleotide probes from the sample by exposing the sample to a dehybridization agent to dehybridize the labeled oligonucleotide probes from the unlabeled oligonucleotide probes. The dehybridization agent can include a chaotropic compound, such as dimethyl sulfoxide and/or formamide. A weight percentage of the chaotropic compound in the dehybridization agent can be 50% or more (e.g., 80% or more).

The hybridization agent can include at least one buffer agent. The hybridization agent can include at least one salt. The hybridization agent can include at least one surfactant. The hybridization agent can include at least one chelating agent. The hybridization agent can include at least one azide-based antibacterial agent.

The dehybridization agent can include at least one buffer agent. The dehybridization agent can include at least one salt. The dehybridization agent can include at least one surfactant. The dehybridization agent can include at least one chelating agent. The dehybridization agent can include at least one azide-based antibacterial agent.

The first composition can include 20 or more different types of unlabeled oligonucleotide probes. The second composition can include 4 or more (e.g., 6 or more) different types of labeled oligonucleotide probes. Each type of unlabeled oligonucleotide probe can include at least 5 bases (e.g., at least 20 bases). Each type of labeled oligonucleotide probe can include at least 5 bases (e.g., at least 10 bases).

Each type of labeled oligonucleotide probe can include a different optical label. The different optical labels can include different fluorescent dyes, different chromogenic moieties, different peptide-containing moieties, different quantum dot-based species, and combinations of any of these.

The second composition can include a first type of labeled oligonucleotide probe among the multiple different types of labeled oligonucleotide probes, the different set of labeled oligonucleotide probes can include the first type of labeled oligonucleotide probe, the at least one image of the biological sample with the multiple different types of labeled oligonucleotide probes bound to the sample can include a first image featuring a measurement signal corresponding to the first type of labeled oligonucleotide probe, the at least one image of the biological sample with the different set of labeled oligonucleotide probes bound to the sample can include a second image featuring a measurement signal corresponding to the first type of labeled oligonucleotide probe, and the methods can include registering the at least one image of the biological sample with the multiple different types of labeled oligonucleotide probes bound to the sample and the at least one image of the biological sample with the different set of labeled oligonucleotide probes bound to the sample based on the measurement signals corresponding to the first type of labeled oligonucleotide probe in the first and second images.

Embodiments of the methods can also include any of the other features described herein, and can include any combinations of features described in connection with different embodiments, unless expressly stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a flow chart showing a series of example steps for RNA detection in a biological sample.

FIG. 2A is a schematic diagram showing hybridization of an unlabeled oligonucleotide probe to a RNA species in a sample.

FIG. 2B is a schematic diagram showing hybridization of a labeled oligonucleotide probe to an unlabeled oligonucleotide probe in a sample.

FIG. 3 is a schematic diagram of an example of a sample imaging system.

FIG. 4 is a flow chart showing a series of example steps for RNA detection in a biological sample.

FIG. 5 is a schematic diagram of an example of a controller.

Like reference symbols in the various drawings indicate like elements.

### DETAILED DESCRIPTION

RNA detection in biological samples provides important information about the sample environment, and a wealth of information concerning cell development and signaling pathways. In situ RNA detection methods are particularly useful, as they can be applied to a wide range of sample types and to samples that are not otherwise amenable to standard laboratory methods.

Conventional RNA detection methods can be limited in various ways. For example, certain detection methods involve hybridization of RNA probes to a sample by temperature cycling (i.e., heating of the sample to induce hybridization, followed by allowing the sample to cool during analysis and/or dehybridization). Unfortunately, repeated adjustment of the sample temperature introduces significant delays in analytical workflows, and accurate temperature control may require relatively complex instrumentation to maintain. Moreover, when the binding efficiency of RNA probes depends on temperature-dependent reactions, quantification of RNA species can be prone to inconsistencies in the analytical workflow.

Certain detection methods involve application of a single type of RNA probe to a sample for detection of a specific RNA species. Using such methods, however, characterization of multiple RNA species in a sample can be a laborious exercise, requiring multiple samples (i.e., a sequence of serial tissue sections) and/or many cycles involving application of a single RNA probe, detection of the applied probe, and removal of the probe following detection.

The present disclosure features methods in which multiple different types of RNA probes can be applied to a single sample. Samples containing RNA species of interest are labeled in a two-step process. First, a sample containing RNA species is exposed to an unlabeled set of oligonucleotide probes. The set of unlabeled oligonucleotide probes consists of oligonucleotides with different sequences. Each unlabeled oligonucleotide probe hybridizes to a different RNA species in the sample with a sequence that is at least partially complementary to the unlabeled oligonucleotide probe. The set of unlabeled oligonucleotide probes can include large numbers of unique probes, and the unlabeled oligonucleotide probes can be added to the sample in a single step (or alternatively, in multiple steps) that precede(s) RNA detection. Following exposure of the sample to the set of unlabeled oligonucleotide probes, a sample with oligonucleotide-labeled RNA species is generated.

Next, the oligonucleotide-labeled RNA species in the sample are exposed to a set of labeled oligonucleotide RNA probes. The labeled oligonucleotide RNA probes each include an optical label. Different types of labeled oligonucleotide RNA probes include distinct (i.e., distinguishable) optical labels. Each different type of labeled oligonucleotide RNA probe hybridizes to a specific oligonucleotide-labeled RNA species. Following hybridization of the labeled oligonucleotide RNA probes to the oligonucleotide-labeled RNA species in the sample, the different labeled oligonucleotide RNA probes can be individually detected and quantified. Because the optical labels are distinct, the sample can be exposed to multiple different types of labeled oligonucleotide RNA probes at the same time, and signals corresponding to multiple different types of labeled oligonucleotide RNA probes can be detected and quantified in a multiplexed manner.

Following detection, the multiple labeled oligonucleotide RNA probes can be removed in a single removal step, either through dehybridization or through other chemical or enzymatic methods. Additional hybridization-detection-dehybridization cycles can be performed with additional batches of labeled oligonucleotide RNA probes on the sample, and in each cycle, multiple different types of labeled oligonucleotide RNA probes can be bound to the sample. In this manner, each cycle can feature multiplexed detection of different labeled oligonucleotide RNA probes.

The methods described herein can optionally be performed entirely or in part under isothermal conditions. As used herein, the term “isothermal” refers to a step or sequence of steps in which the sample temperature is maintained within a temperature range of +/−5 degrees Celsius. In practice, the entire methods or portions of the methods can be performed without either heating or cooling the samples, such that the sample temperature may remain approximately the same as the ambient temperature surrounding the sample.

The methods can be used with a wide variety of biological samples. Examples of such samples include, but are not limited to, fresh samples, frozen samples, fixed-frozen samples, and formalin-fixed, paraffin-embedded (FFPE) samples. In general, such samples correspond to tissue sections, but samples can also be analyzed in other forms as well. For example, samples such as tissue cultures, cell suspensions, cultured cells, whole tumors or biological tissues, tissue biopsies, and cell smears can also be analyzed.

In general, samples can be derived from a variety of sources. In some embodiments, a sample is derived from an animal subject such as a human, a mouse, a rat, a cow, and a pig. In certain embodiments, a sample is derived from a plant. Samples can also be derived from animals or plants that have been modified (i.e., infected or transfected) with genetic material from other sources such as viruses, bacteria, and synthetic sources such as plasmids.

### Analytical Workflow

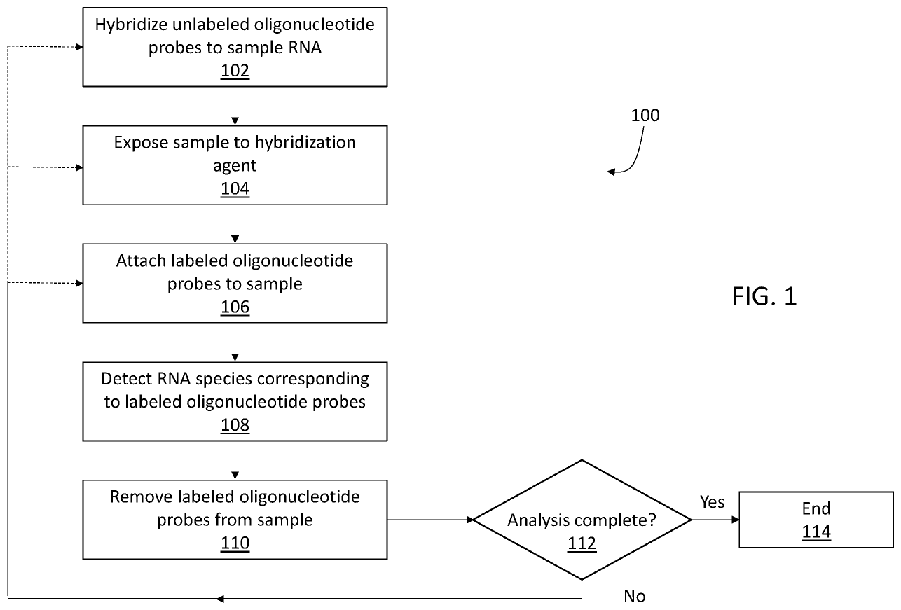

FIG. 1 is a flow chart 100 showing a series of example steps for detecting RNA species in a sample. As a first step 102 in the analysis of the sample, unlabeled oligonucleotide probes are hybridized to RNA species in the sample with sequences that are at least partially complementary to the sequences of the unlabeled oligonucleotide probes. Each unlabeled oligonucleotide probe generally includes between 5 bases and 100 bases (e.g., between 5 bases and 90 bases, between 10 bases and 90 bases, between 10 bases and 80 bases, between 15 bases and 70 bases, between 20 bases and 70 bases, between 20 bases and 50 bases, between 20 bases and 100 bases, or any number of bases within any of these ranges).

In general, an oligonucleotide probe is “unlabeled” if it is not used to directly generate an optical signal for detection of the oligonucleotide probe. In addition to a nucleic acid sequence for hybridization to an RNA species in a sample, unlabeled oligonucleotide probes can generally include a variety of different chemical and biochemical moieties. If none of these additional moieties is used for detection of the oligonucleotide probe, however, the probe is considered “unlabeled” for purpose of the following discussion.

In general, the unlabeled oligonucleotide probes are introduced into the sample by exposing the sample to a composition that includes multiple different types of unlabeled oligonucleotide probes. Each different type of probe selectively hybridizes to a different RNA species or sequence in the sample, thereby “recognizing” the RNA species or sequence. The different types of unlabeled oligonucleotide probes thereby form a set of probes to which the sample of interest is exposed.

A wide variety of different types of unlabeled oligonucleotide probes can be introduced into the sample. In some embodiments, one or more of the oligonucleotide probes includes a nucleic acid sequence as described above. The nucleic acid sequence can be a RNA sequence or a DNA sequence. The sequence can be a natural (e.g., wild type) or synthetic sequence. The nucleic acid sequence can include DNA bases (e.g., A, C, G, T), RNA bases (e.g., A, C, G, U), and any combination for DNA and/or RNA bases. Bases can be conjugated via natural linkages, non-natural (e.g., synthetic) linkages, and a combination of natural and non-natural linkages.

One or more unlabeled oligonucleotide probes can include one or more nucleotides that are capable of base pairing with high reliability with a complementary nucleotide of an RNA species in the sample. Examples of such nucleotides include, but are not limited to, 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine, and 2-thio-uridine.

In certain embodiments, one or more unlabeled oligonucleotide probes can include more than one nucleic acid sequence, and the more than one sequences can be contiguous or non-contiguous in an unlabeled oligonucleotide probe.

Other types of unlabeled oligonucleotide probes can also be used. In some embodiments, for example, one or more unlabeled oligonucleotide probes can include peptide nucleic acids, morpholino, locked, and unlocked nucleic acids, glycol nucleic acids, threose nucleic acids, and peptide-containing species such as aptamers.

In certain embodiments, chemical binding agents such as nucleoside analogs can be used in unlabeled probes. Examples of suitable nucleoside analogs are described in Tor et al., Pure Appl. Chem. 81(2): 263-272 (2009), the entire contents of which are incorporated herein by reference. Other examples of molecular-based binding agents such as small molecule-based agents are described in Cheng et al., Curr. Opin. Struct. Biol. 11(4); 478-484 (2001), Warner et al., Nat. Rev. Drug Discovery 17: 547-558 (2018), Aboul-ela, Future Med. Chem. 2(1): 93-119 (2010), and Yarus, J. Mol. Evol. 47: 109-117 (1998), the entire contents of each of which are incorporated herein by reference.

In the following discussion, it is assumed for simplicity of explanation that the unlabeled oligonucleotide probes contain nucleic acid sequences that selectively bind via hybridization to complementary RNA species in the sample. However, it should be understood that the methods described herein can also be used for any of the above types of unlabeled probes, including probes that are not “oligonucleotide” probes as they do not contain an oligonucleotide sequence.

In step 102, the sample can be exposed to the set of unlabeled oligonucleotide probes in a single step, or alternatively, in multiple (e.g., serial) steps. When the sample is exposed to the unlabeled oligonucleotide probes in multiple steps, the set of probes can be divided into batches, and multiple batches of unlabeled oligonucleotide probes can be applied to the sample in succession, with a washing step between successive exposure steps to remove unbound unlabeled oligonucleotide probes. Alternatively, as will be discussed in further detail below, a first portion of the unlabeled oligonucleotide probes can be applied first to a set of RNA species in the sample and the targeted species can be detected. After detection, the first portion of the unlabeled oligonucleotide probes can optionally be removed from the sample, and a second portion of the unlabeled oligonucleotide probes can be applied to the sample to the same or (more typically) a different set of RNA species. This set of RNA species can be detected, the second portion of the unlabeled oligonucleotide probes can optionally be removed, and one or more additional cycles of application of unlabeled oligonucleotide probes can be performed. In general, there number of applications of unlabeled oligonucleotide probes can be 2 or more (e.g., 3 or more, 4 or more, 5 or more, 6 or more, 7 or more, 8 or more, 9 or more, 10 or more, or even more).

To promote hybridization of the unlabeled oligonucleotide probes to RNA species or sequences in the sample, a hybridization agent can be used. Suitable hybridization agents can include one or more of a variety of different agents, including one or more chaotropic compounds. Examples of suitable hybridization agents are described in greater detail below.

In some embodiments, the unlabeled oligonucleotide probes are bound to the sample under isothermal conditions. Certain conventional RNA detection methods require heating of the sample to induce hybridization of RNA probes to RNA species in situ. In the methods disclosed herein, unlabeled oligonucleotide RNA probes can be hybridized to the RNA sequences or species in the sample without heating the sample, so that the sample remains at the ambient temperature of the environment.

The total number of different types of unlabeled oligonucleotide probes that can be attached to the sample can generally be selected as desired, based on the number of different RNA sequences or species that are to be analyzed. For example, the total number of different types of unlabeled oligonucleotide probes can be 2 or more (e.g., 4 or more, 6 or more, 8 or more, 10 or more, 15 or more, 20 or more, 30 or more, 40 or more, 50 or more, 70 or more, 100 or more, 150 or more, 200 or more, 500 or more, or even more).

In general, an unlabeled oligonucleotide probe will selectively bind to an RNA sequence or species in the sample when the sequence of the unlabeled oligonucleotide probe is at least partially complementary to the RNA sequence or species. As used herein, one nucleic acid sequence is “at least partially complementary” to a second nucleic acid sequence if it contains a contiguous sequence of bases that is complementary to 70% or more of the second nucleic acid sequence.

Unlabeled oligonucleotide probes that hybridize to specific RNA species in the sample can generally be removed by de-hybridization. However, in some embodiments, unlabeled probes bind to RNA species in the sample via other methods. For example, unlabeled oligonucleotide probes can be ligated to specific RNA species in the sample. More generally, unlabeled probes (as discussed above) may not contain oligonucleotide sequences, but may instead include other moieties that selectively bind to RNA species in the sample. The mechanisms that bind these moieties to RNA species may yield associations between the unlabeled probes and RNA species that are reversible (e.g., so that the unlabeled probes can optionally be removed from the sample) or irreversible (e.g., so that the unlabeled probes are bound permanently to the RNA species in the sample).

It should also be noted that combinations of the different types of unlabeled probes can be used in the methods described herein. Further, unlabeled probes can be bound to certain RNA species more than once in a sequence of analytical cycles. For example, in one cycle, a certain RNA species can be selectively hybridized to a first unlabeled oligonucleotide probe which is subsequently removed from the sample. In a succeeding cycle, the RNA species can be bound (i.e., via hybridization or another binding mechanism) to a second unlabeled probe which can be an oligonucleotide probe or a non-oligonucleotide probe. The second unlabeled probe can be bound reversibly or irreversibly to the RNA species.

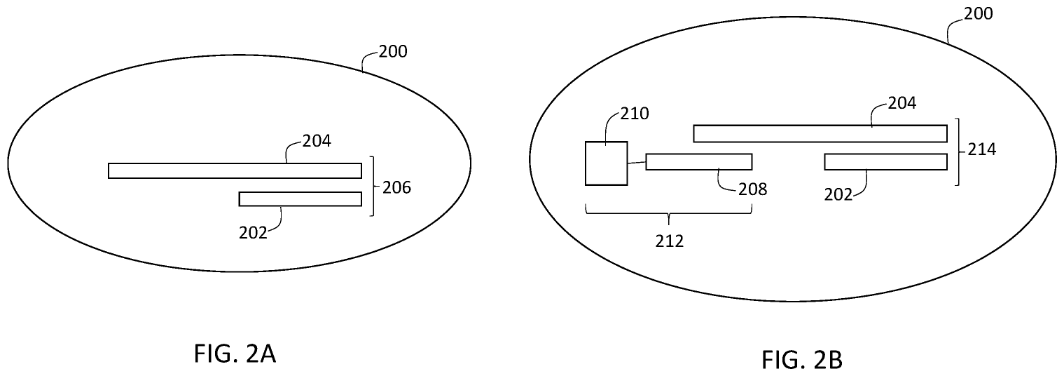

Following exposure of the biological sample to the unlabeled oligonucleotide probes, unhybridized (e.g., unbound) probes are washed out of the sample, e.g., by applying an aqueous washing solution. FIG. 2A is a schematic diagram that illustrates the result of attaching unlabeled oligonucleotide probes to RNA sequences or species in a sample. Sample 200 in FIG. 2A includes a RNA species 202 of interest, to which an unlabeled oligonucleotide probe 204 has been hybridized, as discussed above. As a result of the hybridization, sample 200 includes an oligonucleotide-labeled RNA species 206.

Returning to FIG. 1, after the RNA species in the sample have been selectively hybridized to different unlabeled oligonucleotide probes in step 102, one or more analytical cycles are performed to detect and quantify the RNA species in the sample. As a first step 104 in an analytical cycle, the sample is optionally exposed to a hybridization agent to adjust the hybridization state of the oligonucleotide-labeled RNA species in the sample (and more specifically, to adjust the hybridization state of the unlabeled oligonucleotide probes). In general, the hybridization agent includes one or more substances that prepare the oligonucleotide-labeled RNA species to hybridize to complementary labeled oligonucleotide RNA probes. Suitable examples of hybridization agents are described in greater detail below.

In certain embodiments, the oligonucleotide-labeled RNA species in the sample are exposed to the hybridization agent under isothermal conditions, i.e., without applying heat to the sample. As discussed previously, by exposing the oligonucleotide-labeled RNA species in the sample to the hybridization agent without heating, the analytical workflow can be considerably simplified and accelerated relative to conventional methods that involve temperature cycling.

It should be noted that step 104 is optional. In some embodiments, step 104 can be omitted and oligonucleotide-labeled RNA species in the sample can be directly treated as described in step 106 below.

Next, in step 106, the sample is exposed to a composition that includes multiple different types of labeled oligonucleotide probes. Each labeled oligonucleotide probe generally includes an oligonucleotide conjugated to at least one optical label. The oligonucleotide features a sequence of N bases, where N is typically between 5 and 100 (e.g., between 5 and 90, between 5 and 80, between 10 and 90, between 10 and 80, between 15 and 90, between 15 and 80, between 5 and 70, between 10 and 70, between 20 and 70, between 5 and 50, between 5 and 40, between 5 and 30, between 10 and 50, between 10 and 40, between 20 and 50, between 20 and 40, and any range within the foregoing ranges).

Typically, the composition to which the sample is exposed includes M different types of labeled oligonucleotide probes, where M is 2 or more. For example, M can be 3 or more (e.g., 4 or more, 5 or more, 6 or more, 8 or more, 10 or more, 12 or more, 15 or more, 20 or more, 25 or more, 30 or more, 40 or more, 50 or more, 60 or more, 70 or more, 80 or more, or even more).

Each of the different types of labeled oligonucleotide probes in the composition binds to an unlabeled oligonucleotide probe that is hybridized to an RNA species in the sample. In general, binding between the labeled and unlabeled oligonucleotide probes occurs by hybridization when portions of the probe sequences are at least partially complementary.

FIG. 2B is a schematic diagram that illustrates the result of hybridization between the labeled and unlabeled oligonucleotide probes. Labeled oligonucleotide probe 212 includes an oligonucleotide sequence 208 conjugated to at least one optical label 210. Oligonucleotide sequence 208 hybridizes to a portion of unlabeled oligonucleotide probe 204, forming an optically labeled RNA species 214 in sample 200. Following exposure of the sample to the composition that includes the labeled oligonucleotide probes, unbound labeled probes are washed from the sample (e.g., by applying an aqueous washing solution in one or more cycles).

It should be noted that, in embodiments where the unlabeled probes include a non-oligonucleotide binding moiety that binds to RNA species in the sample as described above, the unlabeled probes can include an oligonucleotide sequence linked to the binding moiety that provides a complementary hybridization site for labeled oligonucleotide probes. Methods for linking oligonucleotide sequences to small molecules, peptides, and other amino acid-based moieties are well known in the art and described, for example, in Drygin et al., Nucl. Acids. Res. 26(21): 4791-4796 (1998), Gampe et al., Angew. Chem. Int. Ed. 55935): 10283 (2016), and Ishizuka et al., Chem. Commun. 88: 10835-10837 (2012), the entire contents of each of which are incorporated herein by reference.

In the example shown in FIG. 2B, the unlabeled oligonucleotide probe 204 binds (e.g., hybridizes) to a single labeled oligonucleotide probe 212. More generally, however, unlabeled oligonucleotide probes 204 can have multiple binding sites for labeled oligonucleotide probes. For example, an unlabeled oligonucleotide probe can include multiple oligonucleotide sequences, each of which binds to a labeled oligonucleotide probe. Such unlabeled oligonucleotide probes can be implemented as branched oligonucleotide structures that include a binding moiety that selectively binds to an RNA species (e.g., any of the binding moieties described above), and multiple oligonucleotide sequences linked to the binding moiety, each of which binds to a labeled oligonucleotide probe. Suitable branched unlabeled oligonucleotide probes can be prepared, for example, as described in Collins et al., Nucl. Acids. Res. 25(15): 2979-2984 (1997), Urdea, Nature Biotechnology 12: 926-928 (1994), and Horn et al., Nucleosides and Nucleotides 8(5-6): 875-877 (1989), the entire contents of each of which are incorporated herein by reference.

Next, in step 108, the optically labeled RNA species in the sample are detected and quantified by detecting optical signals generated by the corresponding optical labels 210. As described above, each of the labeled oligonucleotide probes 212 includes at least one optical label 210. In general, labeled probes can includes a variety of different types of optical labels including—but not limited to—fluorescent species, chromogenic (i.e., absorptive) species, phosphorescent species, and other species that modify incident light (e.g., to generate reflected or transmitted light that differs from the incident light) and/or emit light in response to the incident light.

For purposes of the following discussion, the optical labels conjugated to the labeled probes are referred to as “dyes”, but it should be understood that the term “dyes” refers generally to any of the different types of optical labels described above. As used herein, a “dye” is a moiety that interacts with incident light, and from which emitted light can be measured and used to detect the presence of the dye in a sample. In general, a dye can be a fluorescent moiety, an absorptive moiety (e.g., a chromogenic moiety), or another type of moiety that emits light, or modifies incident light passing through or reflected from a sample where the dye is present so that the presence of the dye can be determined by measuring changes in transmitted or reflected light from the sample.

A wide variety of optical labels or dyes can be conjugated to oligonucleotides to function as labeled oligonucleotide probes that can be used in the methods described herein. Examples of such optical labels include, but are not limited to, small fluorescent or phosphorescent moieties or molecules, proteins (including endogenous and exogenous proteins), peptides and peptide-containing moieties, and quantum dots (e.g., metallic and semi-metallic quantum dots).

In some embodiments, a labeled oligonucleotide probe can include a xanthene-based dye, such as a fluorescein dye and/or a rhodamine dye. Examples of suitable fluorescein and rhodamine dyes include, but are not limited to, fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110.

The dye can also be a cyanine-based dye. Suitable examples of such dyes include, but are not limited to, the dyes Cy3, Cy5 and Cy7. The dye can also be a coumarin dye (e.g., umbelliferone), a benzimide dye (e.g., any of the Hoechst dyes such as Hoechst 33258), a phenanthridine dye (e.g., Texas Red), an ethidium dyes, an acridine dyes, a carbazole dye, a phenoxazine dye, a porphyrin dye, a polymethine dye (e.g., any of the BODIPY dyes), and a quinoline dye.

When the dye is a fluorescent moiety, the dye can be a moiety corresponding to any of the following non-limiting examples and/or derivatives thereof: pyrenes, coumarins, diethylaminocoumarins, FAM, fluorescein chlorotriazinyl, fluorescein, Rl 10, JOE, R6G, tetramethylrhodamine, TAMRA, lissamine, napthofluorescein, Texas Red, Cy3, and Cy5.

In certain embodiments, the dye can include one or more quantum dot-based species. Quantum dot-based fluorophores are available with fluorescence emission spectra in many different spectral bands, and suitable quantum dot-based dyes can be used as labeling species in the methods described herein.

A variety of methods can be used to prepare labeled oligonucleotide probes with dyes. In some embodiments, for example, labeled oligonucleotides probes can include one or more dye-containing bases (which function as the optical labels described above). In certain embodiments, an oligonucleotide sequence of the labeled oligonucleotide probe is linked (e.g., through a linking moiety) to a dye moiety. Labeled oligonucleotide probes that include optical labels can be prepared using a variety of methods such as those described in Ballal et al., J. Biomol. Tech. 20(4): 190-194 (2009), Lawson et al., Scientfic Reports 8: 13970 (2018), Giusti et al., Genome Res. 2: 223-227 (1993), and Smith et al., Meth. Enzymology 155: 260-301 (1987), the entire contents of each of which are incorporated herein by reference.

In general, each of the different types of labeled oligonucleotide probes includes a different type of dye conjugated to the unique oligonucleotide sequence. To detect the different types of labeled oligonucleotide probes bound to the sample, one or more sample images are acquired. Since each of the labeled oligonucleotide probes includes a different dye, separating and quantifying the spectral contributions of the different dyes to the one or more sample images permits identification and quantification of each of the different labeled oligonucleotide probes in the sample. As a consequence of the specific hybridization that occurs between RNA species in the sample and the unlabeled oligonucleotide probes, and between the labeled and unlabeled oligonucleotide probes, different RNA species that are present in the sample can be individually identified and quantified.

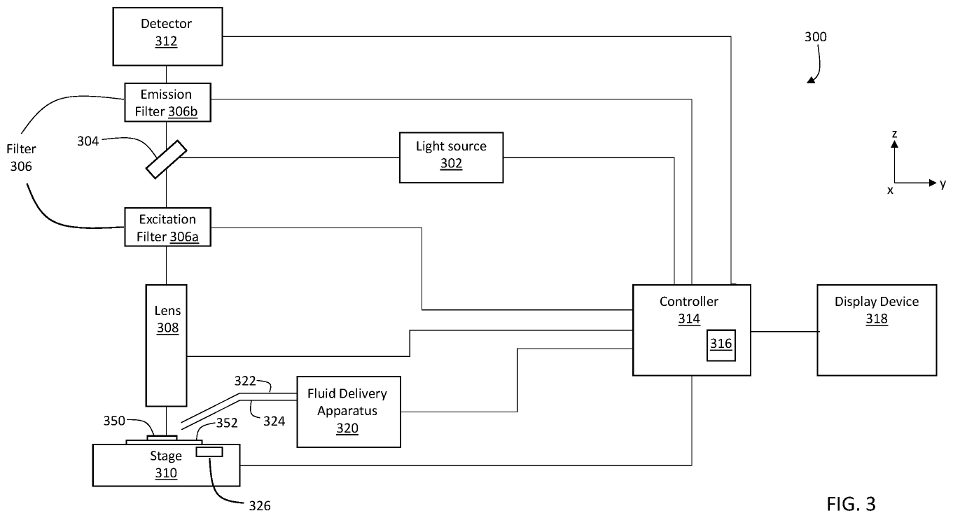

Sample images that include spectral contributions from different dyes of the labeled oligonucleotide probes can be obtained using a variety of different sample imaging systems. An example of one sample imaging system 300, implemented as a microscope (e.g., a fluorescence microscope), is shown in FIG. 3. System 300 includes a light source 302, a dichroic mirror 304, an optical filter 306 (implemented as an excitation filter 306a and an emission filter 306b), an objective lens 308, a stage 310, and a detector 312. Each of these components is coupled to a controller 314 that includes a processing device 316.

Controller 314 transmits and receives control and data signals from each of the system components, and can therefore control each of the components (more specifically, processing device 316 controls each of the system components). In certain embodiments, all of the steps and/or control functions described in connection with system 300 are performed by controller 314. Alternatively, in some embodiments, certain steps can be performed by a human operator of system 300.

Light source 302 is an adjustable source that can produce light having a variable distribution of illumination wavelengths. In some embodiments, for example, light source 302 includes a plurality of LEDs of different wavelengths that can be selectively activated by controller 314 to generate illumination light having desired spectral properties. In certain embodiments, light source 302 includes one or more laser diodes, lasers, incandescent sources, and/or fluorescent sources, each of which is controllable by controller 314.

The illumination light generated by light source 302 reflects from dichroic mirror 304 and is incident on optical filter 306. Filter 306 typically includes multiple filters, each of which can be selectively inserted into the path of the illumination light. Each of the filters has an associated excitation spectral band and one or emission spectral bands. Controller 314 adjusts filter 306 based on the illumination light generated by light source 302 to allow light of a suitable wavelength distribution to be incident on the sample.

Filter 306 can be implemented in various ways. In some embodiments, for example, filter 306 includes a plurality of different epi-filter cubes, each of which can be selectively rotated into the path of the illumination light by controller 314. In certain embodiments, filter 306 includes an adjustable filtering element (e.g., a liquid crystal based element) for which excitation and emission spectral bands can be selectively chosen by controller 314. Other implementations of filter 306 can also be used in system 300.

The filtered illumination light emerging from filter 306 is then focused by lens 308 onto the surface of sample 350, which is supported by a slide 352 that is mounted on a stage 310. Stage 310 permits movement of sample 350 in each of the x- and y-directions, and is controllable by controller 314. Stage 310 can also optionally include a heating element 326 that is controlled by controller 314. Controller 314 can adjust heating element 326 to regulate the temperature of sample 350 when it is positioned on stage 310.

Motion of stage 310 in the x- and y-directions allows the filtered illumination light to be directed to different regions of the sample. By moving the sample relative to the focal region of the illumination light, illumination light can be directed to multiple different regions of the sample, permitting whole-slide imaging of sample 350.

The filtered illumination light generates fluorescence emission from sample 350, and the fluorescence that is emitted in the direction of lens 308 is collimated by lens 308 and passes through filter 306. As discussed above, filter 306—which can be adjusted by controller 314—defines one or more emission spectral bands. The fluorescence emission from sample 350 is filtered by filter 306 such that only light within the one or more emission spectral bands is transmitted by filter 306. The filtered fluorescence emission light is transmitted by dichroic mirror 304 and detected by detector 312.

Detector 312 can be implemented in various ways. In some embodiments, for example, detector 312 includes a CCD-based detection element. In certain embodiments, detector 312 includes a CMOS-based detection element. Detector 312 can also optionally include spectrally-selective optical elements such as one or more prisms, gratings, diffractive elements, and/or filters, to permit wavelength-selective detection of the filtered fluorescence emission light. In response to the incident filtered fluorescence emission light, detector 312 generates one or more electronic signals that represent quantitative measurements of the filtered fluorescence emission light. The signals are transmitted to controller 314 which processes the signals to extract measurement information corresponding to sample 350.

In some embodiments, system 300 includes a fluid delivery apparatus 320 controlled by controller 314. Fluid delivery apparatus 320 optionally delivers fluids—including any of the fluids described herein—to sample 350 on stage 310 via one or more delivery conduits 322, and optionally removes fluids—including any of the fluids described herein—from sample 350. To facilitate fluid delivery and removal, fluid delivery apparatus 320 can optionally include one or more reservoirs for containing fluids, one or more pumps for pressurizing fluids to cause fluid flow, one or more valves for regulating fluid flow, and one or more conduits for transporting fluids within the apparatus. Fluid delivery apparatus 320 can also optionally include other fluid handling and transporting components such as mixing elements, fluid loops, heating elements, cooling elements, and sensors for measuring fluid properties (e.g., optical properties such as fluid spectral properties, fluid absorption properties, and other attributes of fluids). Each of these components can optionally be controlled by controller 314.

Each image obtained from the sample (e.g., using system 300) typically includes contributions from one or more spectral contributors. The spectral contributors can, for example, correspond to the optical labels of the labeled oligonucleotide probes discussed above. For images that substantially include contributions from only a single spectral contributor (e.g., a single labeled oligonucleotide probe that is associated with a single RNA species in the sample), information about the associated RNA species can be obtained directly from the image.

For images that include contributions from multiple spectral contributors (e.g., multiple labeled oligonucleotide probes), information about the multiple RNA species associated with the spectral contributors can be obtained by decomposing the images into a set of component images, each of which includes contributions from substantially only a single spectral contributor and is therefore associated with a single RNA species. Spectral unmixing methods can be used to perform such decompositions, and methods for spectral unmixing are described, for example, in U.S. Pat. No. 7,321,791 and in PCT Patent Application Publication No. WO 2005/040769, the entire contents of each of which are incorporated by reference herein. Controller 314 can optionally implement spectral unmixing and other decomposition methods to analyze the multispectral images obtained by system 300.

As discussed above, the methods described herein permit multiple different types of labeled oligonucleotide probes to be applied to the sample and detected at the same time. Accordingly, the methods allow for identification and quantification of multiple different types of RNA species in the sample in a single analytical cycle. The number of different types of RNA species that can be identified and optionally quantified in a single analytical cycle is M (i.e., the number of different types of labeled oligonucleotide probes that are applied to the sample).

Returning to FIG. 1, following detection of the labeled oligonucleotide probes, the labeled oligonucleotide probes can optionally be removed from the sample in step 110. Labeled oligonucleotide probes can be removed from the sample, for example, in preparation for another analytical cycle involving different labeled oligonucleotide probes, or when the sample is prepared for a different assay (e.g., an assay to detect peptide fragments or genomic DNA in the sample).

In particular, identification and/or quantification of different types of RNA in sample can provide valuable information for subsequent assays that target other types of analytes such as DNA and proteins. Information about the nature and quantity of different types of RNA present in the sample can be used (e.g., by controller 314) to target subsequent assays toward particular types of analytes. For example, an assay for genomic DNA that is performed subsequent to the RNA analysis described herein can be targeted by selecting specific DNA probes for delivery to the sample that correspond to particular types of RNA detected in the sample. Controller 314 (and more generally, system 300) that perform the DNA assay can select specific probes and/or probe pools for delivery to the sample, thereby reducing the quantities of DNA probes that are used. Further, subsequent DNA analysis can be streamlined, as particular DNA sequences can be preferentially analyzed.

As another example, an assay for specific proteins, markers, antibodies, and/or antigens in the sample can be targeted based on information obtained during the RNA analysis described herein. Antibody-based probes for specific proteins and markers can be delivered to the sample by controller 314 based on the nature and quantity of different RNA species in the sample, which are complementary to DNA sequences that code for the proteins and markers. In some embodiments, system 300 can be configured to perform both types of analysis, and controller 314 can use information obtained from RNA analysis to select probes, reagents, and assay conditions for subsequent protein-based and proteomics analyses. Methods for performing such analyses are described for example in PCT Patent Application Publication No. WO 2020/163397, the entire contents of which are incorporated herein by reference.

Various methods can be used to remove labeled oligonucleotide probes from the sample. In some embodiments, for example, chemical cleavage methods (such as reductive disulfide cleavage) can be used. In certain embodiments, enzymatic cleavage methods can be used. Examples of enzymatic cleavage methods for use with nucleic acid species are described in Buckhout-White et al., ACS Omega 3(1): 495-502 (2018), the entire contents of which are incorporated herein by reference.

In certain embodiments, the labeled oligonucleotide probes 212 are removed from the sample via dehybridization from the unlabeled oligonucleotide probes 204. To perform dehybridization, the sample is exposed to a dehybridization agent that includes one or more substances that collectively function to adjust the hybridization state of the unlabeled oligonucleotide probe. The composition of the dehybridization agent is discussed in more detail below. Following dehybridization of the labeled oligonucleotide probes, the free probes are washed from the sample by applying an aqueous washing solution in one or more cycles.

Next, as shown in decision step 112, if detection of RNA species in the sample is complete, the procedure ends at step 114. Alternatively, if detection of RNA species in the sample is not complete, the sample can optionally be subjected to one or more additional analytical cycles. In each additional cycle, a composition that includes one or more (e.g., M in general) different types of labeled oligonucleotide probes can be applied to the sample. Each of the different types of labeled oligonucleotide probes specifically identifies only one type of RNA species in the sample as described above.

One or more additional analytical cycles can be implemented in various ways. In some embodiments, to implement an additional analytical cycle passes to step 104 of flow chart 100, in which the sample is again exposed to a hybridization agent. Subsequently, in step 106, the sample is exposed to a composition that includes multiple different types of labeled oligonucleotide probes, where each labeled oligonucleotide probe generally includes an oligonucleotide conjugated to at least one optical label as discussed above. Each of the different labeled oligonucleotide probes can be different from the labeled oligonucleotide probes to which the sample was exposed in one or more previous cycles. Alternatively, one or more of the labeled oligonucleotide probes can be the same as one or more of the labeled oligonucleotide probes to which the sample was previously exposed in a prior analytical cycle.

In some embodiments, where the sample was previously exposed to a hybridization agent in a previous analytical cycle, an additional analytical cycle bypasses step 104 (i.e., the sample is not exposed to a hybridization agent again) and begins instead at step 106 in FIG. 1.

In certain embodiments, an additional analytical cycle is implemented when control returns from step 112 to step 102 in FIG. 1. Returning to step 102, the sample is again exposed to unlabeled oligonucleotide probes to hybridize the unlabeled probes to RNA species in the sample. During removal step 110, unlabeled oligonucleotide probes 204 can be removed from the sample in addition to labeled oligonucleotide probes 212. Because the unlabeled oligonucleotide probes 204 may be used in subsequent analytical cycles, the population of unlabeled oligonucleotide probes 204 hybridized to RNA species in the sample can be “refreshed” by returning to step 102 and exposing the sample again to unlabeled oligonucleotide probes 204. The types of unlabeled oligonucleotide probes 204 to which the sample is exposed when the workflow returns to step 102 can be same types of unlabeled oligonucleotide probes to which the sample was previously exposed in a prior step 102 of a previous analytical cycle.

Alternatively, in some embodiments, some or all of the unlabeled oligonucleotide probes 204 to which the sample was exposed in the first analytical cycle can be removed from the sample in step 110. Subsequently, after the first analytical cycle is complete, the workflow returns to step 102 and the sample is again exposed to unlabeled oligonucleotide probes 204. However, some or all of the types of unlabeled oligonucleotide probes to which the sample is exposed in the second cycle are different from the types of unlabeled oligonucleotide probes to which the sample was exposed previously. In this manner, in a first analytical cycle (or a first group of multiple analytical cycles), a first set of RNA target species can be interrogated by hybridizing a first set of unlabeled oligonucleotide probes to the first set of RNA target species, and in a second analytical cycle (or a second group of multiple analytical cycles), a second set of RNA target species can be interrogated by hybridizing a second set of unlabeled oligonucleotide probes to the second set of RNA target species. The members of the first and second sets of unlabeled oligonucleotide species can be entirely different, or may have some members (e.g., 1, 2, 3, 4, 5, 6, 7, 8, or even more than 8) members in common.

In some embodiments, among multiple analytical cycles, each type of labeled oligonucleotide probe is hybridized to an unlabeled oligonucleotide probe in the sample and detected in the sample only once. In such embodiments, each type of labeled oligonucleotide probe typically corresponds to a particular RNA target species. Following detection of a measurement signal (e.g., an optical signal) corresponding to the labeled oligonucleotide probe, the presence of the corresponding RNA target species in the sample can be identified and, in certain embodiments, quantified. In subsequent steps of the analytical workflow, the particular RNA target species may not need to be detected and/or quantified again.

In certain embodiments, however, one or more types of labeled oligonucleotide probes are applied to the sample in more than one analytical cycle (e.g., 2 or more, 3 or more, 4 or more, 5 or more, 6 or more, 7 or more, 8 or more, or even more, or even all analytical cycles). By detecting one or more different types of labeled oligonucleotide probes in multiple analytical cycles, the degenerate measured probe signals can function as control signals for other types of probes that are applied and detected only once. For example, signals that are detected for labeled oligonucleotide probes that are applied only once can be normalized or otherwise adjusted based on degenerate labeled oligonucleotide probe signals to ensure that quantitative measurements are accurate. Further, to facilitate co-expression analysis, certain labeled oligonucleotide probe types can be applied multiple times to a sample in combination with other, different types of labeled oligonucleotide probes.

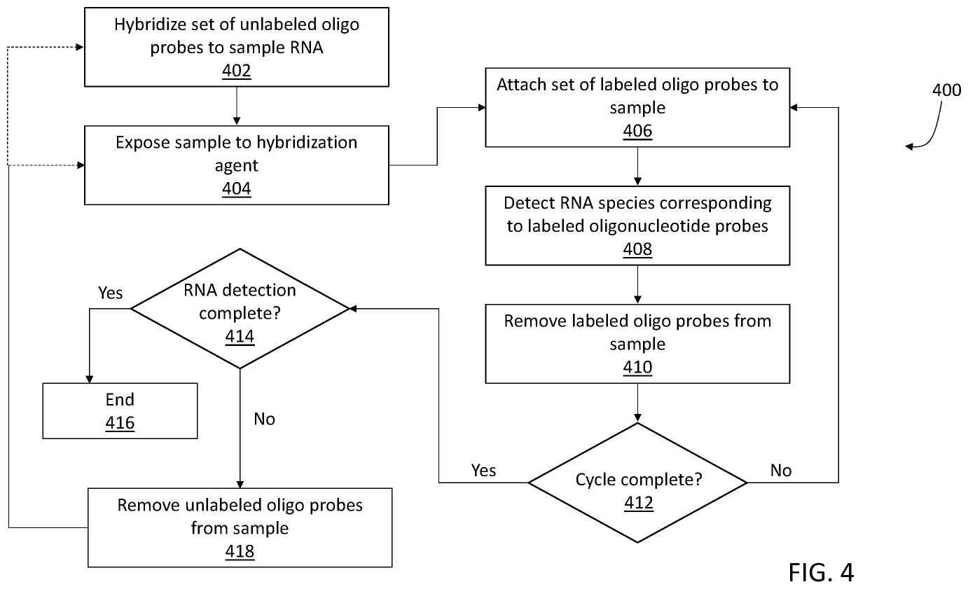

In certain embodiments, the methods described herein can include removal steps for both labeled oligonucleotide probes and unlabeled oligonucleotide probes. FIG. 4 is a flow chart 400 showing a series of example steps for RNA detection in a biological sample in which both labeled and unlabeled oligonucleotide probes can optionally be removed from the sample. In step 402, a sample is exposed to set of unlabeled oligonucleotide probes to bind the probes to RNA species in the sample (e.g., via hybridization) as in step 102 above. Next, in step 404, the sample is optionally exposed to a hybridization agent as in step 104.

A first set of labeled oligonucleotide probes binds to the sample in step 406 as discussed above in connection with step 106, and then in step 408, RNA species in the sample associated with the labeled oligonucleotide probes are detected in the manner discussed above in connection with step 108. Labeled oligonucleotide probes can optionally be removed from the sample in step 410 (as discussed above in connection with step 110), and then control passes to decision step 412. If all desired RNA species targeted by the set of labeled probes from step 406 have been interrogated, the labeling cycle is complete and control passes to step 414. If not control returns to step 406 where a new set of labeled oligonucleotide probes binds to the sample, with or without optional exposure of the sample to a hybridization agent.

In step 414, if all desired RNA species in the sample have been interrogated, the procedure ends at step 416. Alternatively, if additional RNA species remain for analysis, some or all of the unlabeled oligonucleotide probes can optionally be removed from the sample in step 418, after which control returns to either step 402 or 404 as discussed above. To remove unlabeled oligonucleotide probes from the sample, various methods can be used. In some embodiments, for example, unlabeled oligonucleotide probes can be removed from the sample by heating the sample to induce dehybridization (e.g., by activating heating element 326). Alternatively, other methods such as chemical and/or enzymatic methods can be used to remove some or all of the unlabeled oligonucleotide probes.

### Image Registration

In some embodiments, an RNA target species in a sample can be labeled with the same oligonucleotide probe in each analytical cycle so that each image or set of images of the sample obtained in each analytical cycle contains an optical signal corresponding to the RNA target species. In this manner, the optical signals corresponding to the RNA target species function as fiducial markers. The spatial positions of the measured optical signals in each image or set of images can be used to align sample images obtained in each cycle and/or among multiple analytical cycles. To align images in which the measured optical signals appear, a reference position for the fiducial markers is selected (which may correspond, for example, to positions of the fiducial markers in one of the images) and image translations are determined for each of the other images to bring the fiducial markers of each of the other images into alignment with the reference position.

Image registration can also be performed in other ways. In some embodiments, for example, one or more stains can be applied to the sample that specifically localize in certain sample locations. By measuring emission signals from these stains in one or more of a set of images, the set of images can be registered based on features of the sample to which the one or more stains bind. For example, a nuclear stain can be applied to the sample which selectively binds to cell nuclei. When emission signals corresponding to the nuclear stain are measured, the spatial locations of the emission signals indicate the locations of nuclei within the sample. Among multiple images, the spatial locations of the nuclei can be used as fiducial markers to register the multiple images as described above.

While the application of a nuclear stain is discussed by way of example above, a variety of different stains can be applied to the sample and imaged to register sample images. Such stains include, but are not limited to, stains that selectively target cell nuclei, cell membranes, cytoplasm, and cellular organelles.

Alternatively, or in addition, counterstains and other non-specific stains can also be applied to the sample and imaged to register sample images. In general, counterstains and non-specific stains bind to a variety of features in the sample. As such, by imaging a counterstain or non-specific stain in a sample, multiple features of the sample may be visible in the image. Nonetheless, by identifying and selecting one or more of the multiple features in the images, the spatial locations of the selected feature(s) can function as fiducial markers, and can be used to register the images as described above.

### Hybridization and Dehybridization Agents

Various hybridization and dehybridization agents can be used to implement the methods described above. It has been found that to efficiently hybridize and dehybridize RNA probes from biological samples under isothermal conditions, hybridization and dehybridization agents that contain at least one chaotropic compound are particularly effective.

Isothermal hybridization and dehybridization agents generally include one or more chaotropic compounds, and optionally include one or more of the following additional components: one or more surfactants, one or more salts, one or more buffering agents, and one or more chelating agents.

Chaotropic compounds are used to adjust the hybridization state of RNA species in the sample. Various chaotropic compounds can be used in hybridization and dehybridization agents, and such agents can include one or more chaotropic compounds. Examples of suitable chaotropic compounds include, but are not limited to, dimethyl sulfoxide (DMSO), formamide, urea, thiourea, sodium dodecyl sulfate, isopropanol, n-butanol, and guanidinium chloride.

In some embodiments, the percentage by mass of a chaotropic compound in a dehybridization agent can be 50% or more (e.g., 55% or more, 60% or more, 65% or more, 70% or more, 75% or more, 80% or more, 85% or more, 90% or more, 95% or more). In certain embodiments, the percentage by volume of a chaotropic compound in a dehybridization agent can be 50% or more (e.g., 55% or more, 60% or more, 65% or more, 70% or more, 75% or more, 80% or more, 85% or more, 90% or more, 95% or more).

In certain embodiments, where the hybridization agent includes multiple chaotropic compounds, the total percentage by mass of all chaotropic compounds in a dehybridization agent can be 50% or more (e.g., 55% or more, 60% or more, 65% or more, 70% or more, 75% or more, 80% or more, 85% or more, 90% or more, 95% or more). Similarly, where the dehybridization agent includes multiple chaotropic compounds, the total percentage by volume of all chaotropic compounds in a hybridization agent can be 50% or more (e.g., 55% or more, 60% or more, 65% or more, 70% or more, 75% or more, 80% or more, 85% or more, 90% or more, 95% or more).

In general, the percentage by weight and percentage by volume of one or more chaotropic compounds in hybridization agent is smaller than the corresponding percentages in a dehybridization agent. For example, the percentage by mass of a chaotropic compound in a hybridization agent can be 10% or more (e.g., 15% or more, 20% or more, 25% or more, 30% or more, 35% or more, 40% or more) and/or 50% or less (e.g., 45% or less, 40% or less, 35% or less, 30% or less). In certain embodiments, the percentage by volume of a chaotropic compound in a hybridization agent can be 10% or more (e.g., 15% or more, 20% or more, 25% or more, 30% or more, 35% or more, 40% or more) and/or 50% or less (e.g., 45% or less, 40% or less, 35% or less, 30% or less).

In some embodiments, the percentage by mass of a chaotropic compound in a dehybridization agent can be 50% or more (e.g., 55% or more, 60% or more, 65% or more, 70% or more, 75% or more, 80% or more) and/or 99% or less (e.g., 95% or less, 90% or less, 85% or less, 80% or less, 75% or less, 70% or less, 65% or less, 60% or less). In certain embodiments, the percentage by volume of a chaotropic compound in a dehybridization agent can be 50% or more (e.g., 55% or more, 60% or more, 65% or more, 70% or more, 75% or more, 80% or more) and/or 99% or less (e.g., 95% or less, 90% or less, 85% or less, 80% or less, 75% or less, 70% or less, 65% or less, 60% or less).

Where the hybridization agent includes multiple chaotropic compounds, the total percentage by mass of all chaotropic compounds in the hybridization agent can be 10% or more (e.g., 15% or more, 20% or more, 25% or more, 30% or more, 35% or more, 40% or more) and/or 50% or less (e.g., 45% or less, 40% or less, 35% or less, 30% or less). Similarly, where the hybridization agent includes multiple chaotropic compounds, the total percentage by volume of all chaotropic compounds in the hybridization agent can be 10% or more (e.g., 15% or more, 20% or more, 25% or more, 30% or more, 35% or more, 40% or more) and/or 50% or less (e.g., 45% or less, 40% or less, 35% or less, 30% or less).

Where the dehybridization agent includes multiple chaotropic compounds, the total percentage by mass of all chaotropic compounds in the hybridization agent can be 50% or more (e.g., 55% or more, 60% or more, 65% or more, 70% or more, 75% or more, 80% or more) and/or 99% or less (e.g., 95% or less, 90% or less, 85% or less, 80% or less, 75% or less, 70% or less, 65% or less, 60% or less). Similarly, where the dehybridization agent includes multiple chaotropic compounds, the total percentage by volume of all chaotropic compounds in the dehybridization agent can be 50% or more (e.g., 55% or more, 60% or more, 65% or more, 70% or more, 75% or more, 80% or more) and/or 99% or less (e.g., 95% or less, 90% or less, 85% or less, 80% or less, 75% or less, 70% or less, 65% or less, 60% or less).

As noted above, hybridization and dehybridization agents can optionally include one or more surfactants. In general, surfactants function to assist permeation of compounds and agents into the sample, and to facilitate removal of excess/unbound compounds from the sample. Suitable surfactants include, but are not limited to, polyethylene oxide-based surfactants with aromatic hydrocarbon lipophilic or hydrophobic groups such as Triton® X-100, X-114, and X-405 (available from Dow Inc., Midland, Mich.), Tween® 20 and −80, and Brij®-35 and −58 (available from Thermo Fisher, Waltham, Mass.), octyl-beta-glucoside, octylthio glucoside, and sodium dodecyl sulfate. The volume percentage of one or more surfactants in the agents can be between 0.01% and 0.5% (e.g., between 0.02% and 0.4%, between 0.03% and 0.3%, between 0.05% and 0.3%, between 0.1% and 0.5%, between 0.1% and 0.4%, between 0.1% and 0.3%), including any percentage within any of the foregoing ranges such as 0.02%, 0.03%, 0.04%, 0.05%, 0.1%, 0.2%, 0.3%, 0.4%.

Hybridization and dehybridization agents can optionally include one or more salts. Salts function to maintain pH and osmotic conditions within the sample. Suitable examples of such salts include, but are not limited to, NaCl, MgCl₂, CaCl₂), and KCl. The total salt concentration in a hybridization or dehybridization agent can be between 0.01% and 1.0% by mass (e.g., between 0.03% and 0.9%, between 0.03% and 0.8%, between 0.05% and 0.8%, between 0.05% and 0.7%, between 0.1% and 0.6%, between 0.1% and 0.5%), including any percentage within any of the foregoing ranges.

Hybridization and dehybridization agents can optionally include one or more buffering agents to adjust and control the pH of the sample, and maintain appropriate pH conditions for hybridization and dehybridization of RNA probes to/from complementary RNA species in the sample. Suitable buffering agents include, but are not limited to Tris-HCL (available from Sigma-Aldrich, St. Louis, Mo.), Tris-EDTA (available from Thermo Fisher, Waltham, Mass.), and phosphate-buffered saline (PBS). Buffering agents can be present in hybridization and dehybridization agents at concentrations of between 1 mM and 100 mM (e.g., at any concentration within this range).

Hybridization and dehybridization agents can optionally include one or more chelating agents. Examples of suitable chelating agents include, but are not limited to, EDTA, DOTA, benzotriazole, EDDS, EGTA, ethylene diamine, pentetic acid, oxalic acid, sodium citrate, TPEN, tetraphenylporphyrin, TFAC, triethylenetriamine, DTPMP, DIOP, and TTFA. Chelating agents can be present in hybridization and dehybridzation agents at concentrations of between 1 mM and 10 mM, for example, and at any concentration within this range.

In some embodiments, hybridization and dehybridization agents can optionally include one or bacterial growth inhibitors. Examples of bacterial growth inhibitors include, but are not limited to, one or more azide species such as NaN₃.

### Hardware and Software Implementation

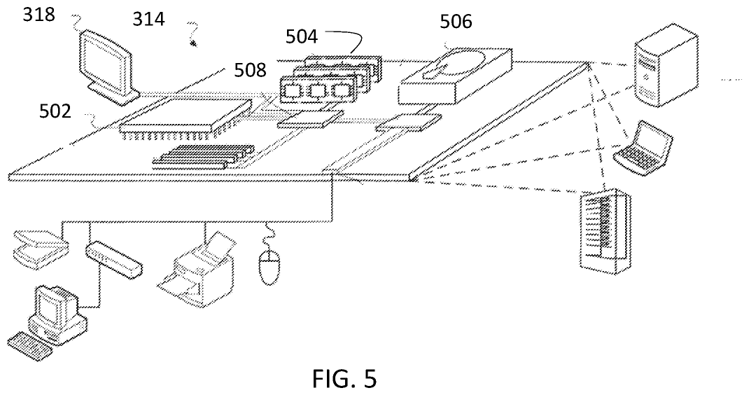

FIG. 5 shows an example of a controller 314, which may be used with the systems and methods disclosed herein. Controller 314 can include one or more processors 502 (e.g., corresponding to processor 316 in FIG. 3), memory 504, a storage device 506 and interfaces 508 for interconnection. The processor 502 can process instructions for execution within the controller 314, including instructions stored in the memory 504 or on the storage device 506. For example, the instructions can instruct the processor 502 to perform any of the analysis and control steps disclosed herein.

The memory 504 can store executable instructions for processor 502, information about parameters of the system such as excitation and detection wavelengths, and measured spectral image information. The storage device 506 can be a computer-readable medium, such as a floppy disk device, a hard disk device, an optical disk device, or a tape device, a flash memory or other similar solid state memory device, or an array of devices, including devices in a storage area network or other configurations. The storage device 506 can store instructions that can be executed by processor 502 described above, and any of the other information that can be stored by memory 504.

In some embodiments, controller 314 can include a graphical processing unit to display graphical information (e.g., using a GUI or text interface) on an external input/output device, such as display device 318. The graphical information can be displayed by a display device (e.g., a CRT (cathode ray tube) or LCD (liquid crystal display) monitor) for displaying any of the information disclosed herein. A user can use input devices (e.g., keyboard, pointing device, touch screen, speech recognition device) to provide input to the controller 314.

The methods disclosed herein can be implemented by controller 314 (and processors 502 and 316) by executing instructions in one or more computer programs that are executable and/or interpretable on the controller 314. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 504, in storage unit 506, and/or on a computer-readable medium, and executed by processor 502 (processor 316) as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

### Applications

The methods, compositions, and systems described herein can be applied to the detection and quantification of a variety of different RNA species in a biological sample. For example, RNA species within a sample that can be detected include messenger RNA (mRNA), transfer RNA (tRNA), ribosomal RNA (rRNA), small nuclear RNA (snRNA), transfer-messenger RNA (tmRNA), small nucleolar RNA (snoRNA), guide RNA (gRNA), antisense RNA (aRNA), CRISPR RNA (crRNA), long non-coding RNA (lncRNA), microRNA (miRNA), small interfering RNA (siRNA), short hairpin RNA (shRNA), trans-acting siRNA (tasiRNA), and enhancer RNA (eRNA).

Other types of RNA-containing species can also be analyzed. For example, where a sample is labeled with a RNA-containing tagging agent (such as an RNA nucleotide sequence conjugated to an antibody that selectively binds to corresponding antigens in the sample), the RNA-based tagging agents can be detected and quantified using the methods described herein. A wide variety of different types of RNA-based tagging agents can be analyzed in this manner.

### OTHER EMBODIMENTS

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

1. A method, comprising:
(a) contacting a biological sample with a first composition comprising multiple different types of unlabeled oligonucleotide probes that hybridize to RNA species in the sample;
(b) contacting the biological sample with a hybridization agent comprising a chaotropic compound;
(c) contacting the biological sample with a second composition comprising multiple different types of labeled oligonucleotide probes, wherein each of the different types of labeled oligonucleotide probes selectively hybridizes to one of the different types of unlabeled oligonucleotide probes;
(d) obtaining at least one image of the biological sample with the multiple different types of labeled oligonucleotide probes bound to the sample; and
(e) identifying spatial locations of the RNA species in the sample based on components of the at least one image that correspond to the different types of labeled oligonucleotide probes,
wherein the biological sample is contacted with the second composition under isothermal conditions.

2. The method of claim 1, further comprising:
(f) removing the labeled oligonucleotide probes from the sample.

3. The method of claim 2, further comprising repeating steps (c)-(e) with a different set of labeled oligonucleotide probes to identify spatial locations of at least one additional RNA species in the sample.

4. The method of claim 3, further comprising repeating step (b) prior to repeating steps (c)-(e).

5. The method of claim 3, further comprising, prior to repeating steps (c)-(e):
contacting the biological sample with a third composition comprising multiple different types of unlabeled oligonucleotide probes that hybridize to RNA species in the sample,
wherein the multiple different types of unlabeled oligonucleotide probes of the third composition are also present in the first composition.

6. The method of claim 1, wherein the chaotropic compound comprises dimethylsulfoxide.

7. The method of claim 1, wherein the chaotropic compound comprises formamide.

8. The method of claim 1, wherein a weight percentage of the chaotropic compound in the hybridization agent is between 5% and 20%.

9. The method of claim 2, further comprising removing the labeled oligonucleotide probes from the sample by reductive cleavage.

10. The method of claim 2, further comprising removing the labeled oligonucleotide probes from the sample by enzymatic cleavage.

11. The method of claim 2, further comprising removing the labeled oligonucleotide probes from the sample by exposing the sample to a dehybridization agent to dehybridize the labeled oligonucleotide probes from the unlabeled oligonucleotide probes.

12. The method of claim 11, wherein the dehybridization agent comprises a chaotropic compound.

13. The method of claim 12, wherein the chaotropic compound of the dehybridization agent comprises at least one member selected from the group consisting of dimethyl sulfoxide and formamide.

14. (canceled)

15. The method of claim 12, wherein a weight percentage of the chaotropic compound in the dehybridization agent is 50% or more.

16. (canceled)

17. The method of claim 1, wherein the hybridization agent comprises at least one member selected from the group consisting of a buffer agent, a salt, and a surfactant.

18-19. (canceled)

20. The method of claim 1, wherein the hybridization agent comprises at least one chelating agent.

21. The method of claim 1, wherein the hybridization agent comprises at least one azide-based antibacterial agent.

22. The method of claim 12, wherein the dehybridization agent comprises at least one member selected from the group consisting of a buffer agent, a salt, and a surfactant.

23-24. (canceled)

25. The method of claim 12, wherein the dehybridization agent comprises at least one chelating agent.

26. The method of claim 12, wherein the dehybridization agent comprises at least one azide-based antibacterial agent.

27. The method of claim 1, wherein the first composition comprises 20 or more different types of unlabeled oligonucleotide probes.

28. The method of claim 1, wherein the second composition comprises 4 or more different types of labeled oligonucleotide probes.

29. (canceled)

30. The method of claim 1, wherein each type of unlabeled oligonucleotide probe comprises at least 5 bases.

31. (canceled)

32. The method of claim 1, wherein each type of labeled oligonucleotide probe comprises at least 5 bases.

33. (canceled)

34. The method of claim 1, wherein each type of labeled oligonucleotide probe comprises a different optical label.

35. The method of claim 34, wherein the different optical labels comprise at least one member selected from the group consisting of different fluorescent dyes, different chromogenic moieties, and different quantum dot-based species.

36. (canceled)

37. The method of claim 34, wherein the different optical labels comprise different peptide-containing moieties.

38. (canceled)

39. The method of claim 3, wherein:
the second composition comprises a first type of labeled oligonucleotide probe among the multiple different types of labeled oligonucleotide probes;
the different set of labeled oligonucleotide probes comprises the first type of labeled oligonucleotide probe;
the at least one image of the biological sample with the multiple different types of labeled oligonucleotide probes bound to the sample comprises a first image comprising a measurement signal corresponding to the first type of labeled oligonucleotide probe;
the at least one image of the biological sample with the different set of labeled oligonucleotide probes bound to the sample comprises a second image comprising a measurement signal corresponding to the first type of labeled oligonucleotide probe; and
the method further comprises registering the at least one image of the biological sample with the multiple different types of labeled oligonucleotide probes bound to the sample and the at least one image of the biological sample with the different set of labeled oligonucleotide probes bound to the sample based on the measurement signals corresponding to the first type of labeled oligonucleotide probe in the first and second images.
